(19) United States
(12) Patent Application Publication
(10) Pub. No.: US 2026/0311800 A1
(43) Pub. Date: Oct. 8, 2026

(54) PROGRAMMABLE E3 LIGASE IDENTIFICATION

(71) Applicant: MONTE ROSA THERAPEUTICS AG, Basel (CH)

(72) Inventors: Pablo Gainza, Lausanne (CH); Jennifer Han-Chun Tsai, Boston, MA (US); Ramon Miguel Caceres, Needham, MA (US)

(21) Appl. No.: 19/484,441

(22) PCT Filed: May 16, 2024
(86) PCT No.: PCT/US2024/029650

§ 371 Date: Nov. 14, 2025

Related U.S. Application Data
(60) Provisional application No. 63/467,239, filed on May 17, 2023.

(51) Int. Cl. G16B 15/30; G16B 40/00

(52) U.S. Cl. CPC G16B 15/30; G16B 40/00

(57) ABSTRACT

Described herein are compositions, methods, systems, and apparatus, including computer programs encoded on a computer storage medium for, among other things, identification of programmable E3 ligases.

### PRIORITY

This application claims the benefit of U.S. Provisional Application Ser. No. 63/467,239, filed on May 17, 2023. The entire contents of the foregoing are incorporated herein by reference.

### TECHNICAL FIELD

Described herein are methods and systems useful, for example, for identification and characterization of protein interaction domains, and also, for example, for predicting, identifying, classifying, and selecting E3 ligases.

### BACKGROUND

Protein biosynthesis and degradation is a dynamic process which sustains normal cell homeostasis. The ubiquitin-proteasome system is a master regulator of protein homeostasis, by which proteins are initially targeted for poly-ubiquitination by E3 ligases and then degraded into short peptides by the proteasome. This system can be modulated, for example, by molecular glues. A need exists for the development of methods for identification and characterization of programmable E3 ligases.

### SUMMARY

The E3 ubiquitin ligase complex ubiquitinates many other proteins and can be manipulated with small molecules to trigger targeted degradation of specific substrate proteins of interest, including proteins that are not naturally targeted for degradation. Binding of substrate proteins with the E3 ubiquitin ligase complex is permitted if certain features, known as degrons, are present on the substrate proteins.

In some cases, binding of small molecules (e.g., molecular glues) to E3 ligase substrate receptors such as cereblon (CBRN) modulates the substrate selectivity of the complex, e.g., by changing the molecular surface of the E3 ligase substrate receptor protein, effectively hijacking the innate in vivo protein degradation system in order to SUBSTITUTE SHEET (RULE 26) degrade specific target proteins, e.g., for therapeutic effect (sometimes referred to as targeted protein degradation).

Molecular glues stabilize protein-protein interactions (e.g., between an E3 ligase substrate receptor protein and a neosubstrate), and, in cases where they lead to degradation of the neosubstrate, they are known as molecular glue degraders. Molecular glue degraders are a recently discovered therapeutic modality, with several clinically approved drugs (e.g. indisulam and lenalidomide), whose targets would have been otherwise considered undruggable. Molecular glue degraders have the potential to become the only modality capable of downregulating the large fraction of the proteome (>75%) considered undruggable using other approaches.

This raises the challenge of identifying neosubstrates and/or neosurfaces, in effect matching targets to particular E3 ligases, given a known or a yet unknown molecular glue. Thus, a critical need exists to identify programmable E3 ligase substrate surface receptors, as well their corresponding neosubstrates. Thus, described herein are, among other things, methods for the identification of E3 ligase substrate receptor proteins and substrate proteins capable of being targeted by the E3 ligase machinery, based on the protein molecular surface (quinary) representations of the protein structure(s). The methods are useful, for example, in matching E3 ligases (e.g., an E3 ligase substrate receptor protein such as CRBN) to degrons (e.g., in target proteins), in the presence or absence of a molecular glue.

The system can reduce consumption of time and laboratory resources, e.g., assays and personnel-hours, compared to experimentally determining neosubstrates and/or neosurfaces for particular E3 ligases. Experimentally determining neosubstrates and/or neosurfaces can be prohibitively complex, uncertain, resource-intensive, and time-consuming since it can involve synthesizing many versions of a potential substrate for each E3 ligase as part of an exhaustive search.

The methods described herein provide, for the first time, the identification of programmable E3 ligase substrate receptor proteins, as well as corresponding neosubstrates, based on their surface features. The methods described herein are useful, for example, to identify programmable E3 ligase substrate receptor proteins and degrons in the neosubstrates of the same, independently of their underlying primary sequence and secondary structure, based on, for example, surface similarity and/or complementarity.

Provided herein are methods for calculating a programmability score for a known or suspected E3 ligase substrate receptor protein, the method comprising: a) providing data comprising a first set of structural features, optionally molecular surface features representing a putative interaction region on a protein known or suspected to be an E3 ligase substrate receptor protein, optionally a protein listed in Table 1; and, based on the first set of structural features, calculating a PPI propensity score for the protein, optionally using a machine learning algorithm trained on structural features of known or predicted protein-protein interaction surfaces; b) providing data comprising a second set of structural features, optionally molecular surface features representing a putative pocket region on the protein, and, based on the second set of structural features, calculating a ligandability score for the protein, optionally using a machine learning algorithm trained on structural features of known or predicted high affinity small molecule pockets; c) based on the PPI propensity score and the ligandability score, calculating a programmability score for the known or suspected E3 ligase substrate receptor protein; d) optionally identifying the presence or absence of a cysteine residue within the putative pocket region; and e) optionally, classifying the E3 ligase substrate receptor protein as programmable or not based on the programmability score and optionally the presence or absence of a cysteine residue within the putative pocket region.

In some embodiments, calculating a PPI propensity score comprises: generating an embedding for the putative interaction region by processing the data comprising the first set of structural features using an embedding neural network to generate an embedding of the putative interaction region in an embedding space; and determining, for the putative interaction region, the likelihood of being involved in a protein-protein interaction.

In some embodiments, calculating a ligandability score comprises: generating an embedding for the putative pocket region by processing the data comprising the first set of structural features using an embedding neural network to generate an embedding of the putative pocket region in an embedding space; and determining, for the putative pocket region, the likelihood of being a ligand binding pocket.

In some embodiments, classifying comprises comparing the programmability score to a threshold and, if a) the threshold is satisfied and b) optionally if a cysteine is present, classifying the E3 ligase substrate receptor protein as programmable; else, classifying the E3 ligase substrate receptor protein as not programmable.

In some embodiments, the threshold is a programmability score for a known E3 ligase substrate receptor protein, optionally CRBN.

Also described herein are methods for calculating a desirability score for a known or suspected E3 ligase substrate receptor protein, comprising: a) providing data comprising a first set of structural features, optionally molecular surface features representing a putative interaction region on a protein known or suspected to be an E3 ligase substrate receptor protein, optionally a protein listed in Table 1; and, based on the first set of structural features, calculating a PPI propensity score for the protein, optionally using a machine learning algorithm trained on structural features of known or predicted protein-protein interaction surfaces; b) providing data comprising a second set of structural features, optionally molecular surface features representing a putative pocket region on the protein, and, based on the second set of structural features, calculating a ligandability score for the protein, optionally using a machine learning algorithm trained on structural features of known or predicted high affinity small molecule pockets; c) calculating an activity score for the protein; d) calculating an essentialness score for the protein; e) calculating a cytoplasm score for the protein; f) calculating an expression score for the protein; and c) based on the PPI propensity score, the ligandability score, the activity score, the essentialness score, and the cytoplasm score, calculating a desirability score for the known or suspected E3 ligase substrate receptor protein; d) optionally identifying the presence or absence of a cysteine residue within the putative pocket region; and e) optionally, classifying the E3 ligase substrate receptor protein as desirable or not based on the desirability score and optionally the presence or absence of a cysteine residue within the putative pocket region.

In some embodiments, calculating a PPI propensity score comprises: generating an embedding for the putative interaction region by processing the data comprising the first set of structural features using an embedding neural network to generate an embedding of the putative interaction region in an embedding space; and determining, for the putative interaction region, the likelihood of being involved in a protein-protein interaction.

In some embodiments, calculating a ligandability score comprises: generating an embedding for the putative pocket region by processing the data comprising the first set of structural features using an embedding neural network to generate an embedding of the putative pocket region in an embedding space; and determining, for the putative pocket region, the likelihood of being a ligand binding pocket.

In some embodiments, calculating an activity score for the protein comprises assigning a numeric value based on the count of known interactions. In some embodiments, the numeric value assigned is 0.0 if the count is 0, 0.1 if the count is >0 and <=10, and 0.2 if the count is >10.

In some embodiments, calculating an essentialness score for the protein comprises classifying the protein as essential or not, assigning a value of 0 if essential and 1 if not essential, and optionally applying a penalty. In some embodiments, applying a penalty comprises multiplying by a factor of from 0.0 to 0.3, optionally 0.1.

In some embodiments, calculating a cytoplasm score for the protein comprises classifying the protein as known to be present in the cytoplasm or not, assigning a value of 0 if not known to be in the cytoplasm and 1 if known to be in the cytoplasm, and optionally applying a penalty. In some embodiments, applying a penalty by multiplying by a factor of from 0.0 to 0.3, optionally 0.1.

In some embodiments, calculating an expression score comprises assigning a numeric value based on the level of known expression. In some embodiments, the numeric value assigned is 0.4 if the expression level is high, 0.2 if the expression level is medium, 0.1 if the expression level is low, and 0 if expression is not detected. In some embodiments, the level of known expression is the average of expression in all tissues.

In some embodiments, classifying comprises comparing the desirability score to a threshold and, if a) the threshold is satisfied and b) optionally if a cysteine is present, classifying the E3 ligase substrate receptor protein as desirable; else, classifying the E3 ligase substrate receptor protein as not desirable. In some embodiments, the threshold is a programmability score for a known E3 ligase substrate receptor protein, optionally CRBN.

Also provided herein are methods for identifying protein(s) and/or protein domain(s) predicted to interact with an E3 ligase substrate receptor protein surface or neosurface, optionally an E3 ligase substrate receptor protein listed in Table 1, optionally an E3 ligase substrate receptor identified by a method described herein, the method comprising: a) providing data comprising structural features, optionally molecular surface features of a plurality of surface patches, each of which represents a region on a respective protein molecular surface; and, based on the structural features, identifying a first set of protein(s) comprising surfaces similar to the surface or neosurface of a known or suspected E3 ligase substrate receptor protein, using a machine learning algorithm trained on structural features of interacting pairs of protein surface patches; b) identifying a second set of protein(s) known or suspected to interact with the first set of protein(s), and optionally identifying a set of protein domain(s) representing the second set of protein(s); d) optionally filtering the second set of protein(s) or the set of protein domain(s); and e) determining that one or more of second set of protein(s), the filtered second set of protein(s), the set of protein domain(s), or the set of filtered protein domain(s) is predicted to interact with the known or suspected E3 ligase substrate receptor protein surface or neosurface.

In some embodiments, identifying a first set of protein(s) comprises: generating embeddings for each of the surface patches by processing the data comprising the set of surface features using an embedding neural network to generate an embedding of the surface patches in an embedding space; and determining, for the surface patch, a surface/neosurface similarity score; and based on the surface/neosurface score, including or excluding the corresponding protein from the first set.

In some embodiments, including or excluding is based on a rank and/or a threshold score.

In some embodiments, identifying a second set of protein(s) comprises searching a database.

In some embodiments, the molecular surface features comprise geometric and/or chemical features.

In some embodiments, the geometric features are selected from the group consisting of shape index, distance-dependent curvature, geodesic polar coordinates, radial (angular) coordinates, and combinations thereof.

In some embodiments, the chemical features are selected from the group consisting of hydropathy index, continuum electrostatics, location of free electrons, location of free proton donors, and combinations thereof.

In some embodiments, the machine learning algorithm comprises a geometric deep learning model.

In some embodiments, the geometric deep learning model is a three-dimensional convolutional neural network.

In some embodiments, the method is performed at least in part by one or more computers.

In some embodiments, the method further comprises testing or having tested the E3 ligase substrate receptor protein or protein(s) and/or protein domain(s) predicted to interact with an E3 ligase substrate receptor protein surface or neosurface in an E3 ligase substrate detection assay, with or without a binding modulator.

In some embodiments, the E3 ligase substrate detection assay is selected from the group consisting of a proximity assay, a binding assay, and a degradation assay.

Also provided herein are systems comprising: one or more computers; and
one or more storage devices communicatively coupled to the one or more computers, wherein the one or more storage devices store instructions that, when executed by the one or more computers, cause the one or more computers to perform operations comprising the method of any one of the preceding embodiments.

Also provided herein are one or more non-transitory computer storage media storing instructions that when executed by one or more computers cause the one or more computers to perform operations comprising the method of any one of the preceding embodiments.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Throughout this specification, an “embedding” refers to an ordered collection of numerical values, e.g., a vector, matrix, or other tensor of numerical values.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows an example of a method for programmable E3 ligase identification.

FIG. 2 shows an example of a method for ranking E3 ligases (e.g., by desirability score).

### DETAILED DESCRIPTION

Described herein are methods and compounds useful, for example, for predicting, identifying, classifying, and selecting programmable E3 ligase substrate receptor proteins and, optionally their corresponding neosubstrates, using, for example, molecular surface features of protein(s). The molecular surface is a higher-level representation of protein structure than protein structure or sequence and the methods described herein provide an improvement, for example, over methods utilizing lower level representation(s) of protein structure.

### E3 Ligases and E3 Ligase Substrate Receptors

E3 ligases recognize protein substrates and, when complexed with E2 conjugating enzymes loaded with ubiquitin, results in ubiquitination of the protein. E3 ligases and their substrate receptor proteins are known and described in the art, for example, in Yang et al., “E3 Ubiquitin Ligases: Styles, Structures, and Functions,” Mol Biomed doi: 10.1186/s43556-021-00043-2 (2021).

Cereblon (CRBN), for example, forms an E3 ubiquitin ligase complex with damaged DNA binding protein 1 (DDB1), Cullin-4A (CUL4A), and regulator of cullins 1 (ROC1).

In some cases, the ligase substrate receptor protein is an E3 ligase substrate receptor protein identified in the following table.

TABLE 1
Examples of E3 ligase substrate receptor proteins

| UniProt ID | Gene Name | E3 Ligase Type |
|---|---|---|
| O15033 | AREL1 | HECT |
| Q66K89 | E4F1 | HECT |
| Q09472 | EP300 | HECT |
| Q7L622 | G2E3 | HECT |
| Q8IYU2 | HACE1 | HECT |
| Q9ULT8 | HECTD1 | HECT |
| Q5U5R9 | HECTD2 | HECT |
| Q5T447 | HECTD3 | HECT |
| Q76N89 | HECW1 | HECT |
| Q9P2P5 | HECW2 | HECT |
| Q15751 | HERC1 | HECT |
| O95714 | HERC2 | HECT |
| Q15034 | HERC3 | HECT |
| Q5GLZ8 | HERC4 | HECT |
| Q9U114 | HERC5 | HECT |
| Q8IVU3 | HERC6 | HECT |
| Q7Z6Z7 | HUWE1 | HECT |
| Q96J02 | ITCH | HECT |
| Q9UDY8 | MALT1 | HECT |
| P46934 | NEDD4 | HECT |
| Q96PU5 | NEDD4L | HECT |
| Q9HCE7 | SMURF1 | HECT |
| Q9HAU4 | SMURF2 | HECT |
| Q7Z3V4 | UBE3B | HECT |
| O95071 | UBR5 | HECT |
| Q9H0M0 | WWP1 | HECT |
| O00308 | WWP2 | HECT |
| Q96JP5 | ZFP91 | HECT |
| Q9UKV5 | AMFR | RING |
| Q99728 | BARD1 | RING |
| Q9UIG0 | BAZ1B | RING |
| Q9NZS9 | BFAR | RING |
| Q13490 | BIRC2 | RING |
| Q13489 | BIRC3 | RING |
| Q96CA5 | BIRC7 | RING |
| Q96P09 | BIRC8 | RING |
| P35226 | BMI1 | RING |
| Q7Z569 | BRAP | RING |
| P38398 | BRCA1 | RING |
| P22681 | CBL | RING |
| Q13191 | CBLB | RING |
| Q9ULV8 | CBLC | RING |
| Q75N03 | CBLL1 | RING |
| Q8N7E2 | CBLL2 | RING |
| Q9NPC3 | CCNB1IP1 | RING |
| Q96EP1 | CHFR | RING |
| O95628 | CNOT4 | RING |
| Q86Y01 | DTX1 | RING |
| Q86UW9 | DTX2 | RING |
| Q8N919 | DTX3 | RING |
| Q8TDB6 | DTX3L | RING |
| Q9Y2E6 | DTX4 | RING |
| Q86Y13 | DZIP3 | RING |
| Q9NW38 | FANCL | RING |
| Q9H116 | GZF1 | RING |
| Q14527 | HLTF | RING |
| Q8IU81 | IRF2BP1 | RING |
| Q8NB78 | KDM1B | RING |
| Q8NEZ4 | KMT2C | RING |
| Q99732 | LITAF | RING |
| Q8TBB1 | LNX1 | RING |
| Q8N448 | LNX2 | RING |
| Q1L5Z9 | LONRF2 | RING |
| Q496Y0 | LONRF3 | RING |
| Q6UWE0 | LRSAM1 | RING |
| O94822 | LTN1 | RING |
| Q13233 | MAP3K1 | RING |
| Q8TCQ1 | MARCHF1 | RING |
| Q8NA82 | MARCHF10 | RING |
| A6NNE9 | MARCHF11 | RING |
| Q9P0N8 | MARCHF2 | RING |
| Q86UD3 | MARCHF3 | RING |
| Q9P2E8 | MARCHF4 | RING |
| Q9NX47 | MARCHF5 | RING |
| O60337 | MARCHF6 | RING |
| Q9H992 | MARCHF7 | RING |
| Q5T0T0 | MARCHF8 | RING |
| Q86YJ5 | MARCHF9 | RING |
| Q00987 | MDM2 | RING |
| O15151 | MDM4 | RING |
| Q5U5Q3 | MEX3C | RING |
| Q86XN8 | MEX3D | RING |
| O60291 | MGRN1 | RING |
| Q86YT6 | MIB1 | RING |
| Q96AX9 | MIB2 | RING |
| O15344 | MID1 | TRIM |
| Q9UJV3 | MID2 | TRIM |
| Q9UHC7 | MKRN1 | RING |
| Q13064 | MKRN3 | RING |
| P51948 | MNAT1 | RING |
| Q9HCI7 | MSL2 | RING |
| Q969V5 | MUL1 | RING |
| 075592 | MYCBP2 | RING |
| Q8WY64 | MYLIP | RING |
| O76050 | NEURL1 | RING |
| A8MQ27 | NEURL1B | RING |
| Q96EH8 | NEURL3 | RING |
| Q12986 | NFX1 | RING |
| Q6VVB1 | NHLRC1 | RING |
| Q8WV22 | NSMCE1 | RING |
| Q96MF7 | NSMCE2 | RING |
| Q5W0B1 | OBI1 | RING |
| Q86WC4 | OSTM1 | RING |
| Q9BSM1 | PCGF1 | RING |
| P35227 | PCGF2 | RING |
| Q3KNV8 | PCGF3 | RING |
| Q86SE9 | PCGF5 | RING |
| Q9BYE7 | PCGF6 | RING |
| Q9UPQ7 | PDZRN3 | RING |
| Q96FA3 | PELI1 | RING |
| Q9HAT8 | PELI2 | RING |
| O60683 | PEX10 | RING |
| O00623 | PEX12 | RING |
| P28328 | PEX2 | RING |
| O94880 | PHF14 | RING |
| Q96BD5 | PHF21A | RING |
| Q9BWX1 | PHF7 | RING |
| Q9P1Y6 | PHRF1 | RING |
| O75925 | PIAS1 | RING |
| O75928 | PIAS2 | RING |
| Q9Y6X2 | PIAS3 | RING |
| Q8N2W9 | PIAS4 | RING |
| Q8NG27 | PJA1 | RING |
| O43164 | PJA2 | RING |
| P29590 | PML | RING |
| O43663 | PRC1 | RING |
| Q9NS91 | RAD18 | RING |
| P15918 | RAG1 | RING |
| Q13702 | RAPSN | RING |
| Q7Z6E9 | RBBP6 | RING |
| Q9H1K0 | RBSN | RING |
| P62877 | RBX1 | RING |
| Q96PM5 | RCHY1 | RING |
| Q8WZ73 | RFFL | RING |
| Q6ZWI9 | RFPL4B | RING |
| Q6PCD5 | RFWD3 | RING |
| Q6R327 | RICTOR | RING |
| Q06587 | RING1 | RING |
| Q9NVW2 | RLIM | RING |
| Q9H871 | RMND5A | RING |
| Q8N5U6 | RNF10 | RING |
| O00237 | RNF103 | RING |
| Q9Y3C5 | RNF11 | RING |
| Q6ZNA4 | RNF111 | RING |
| O15541 | RNF113A | RING |
| Q9Y508 | RNF114 | RING |
| Q9Y4L5 | RNF115 | RING |
| Q9H9V4 | RNF122 | RING |
| Q96EQ8 | RNF125 | RING |
| Q9BV68 | RNF126 | RING |
| Q8TEB7 | RNF128 | RING |
| O43567 | RNF13 | RING |
| Q86XS8 | RNF130 | RING |
| Q8WVZ7 | RNF133 | RING |
| Q8IUD6 | RNF135 | RING |
| Q8WVD3 | RNF138 | RING |
| Q8WU17 | RNF139 | RING |
| Q8WVD5 | RNF141 | RING |
| Q96MT1 | RNF145 | RING |
| Q9NTX7 | RNF146 | RING |
| Q8N7C7 | RNF148 | RING |
| Q8NC42 | RNF149 | RING |
| Q9ULK6 | RNF150 | RING |
| Q6ZSG1 | RNF165 | RING |
| Q96A37 | RNF166 | RING |
| Q9H6Y7 | RNF167 | RING |
| Q81YW5 | RNF168 | RING |
| Q8NCN4 | RNF169 | RING |
| Q96K19 | RNF170 | RING |
| Q86T96 | RNF180 | RING |
| Q9P0P0 | RNF181 | RING |
| Q8N6D2 | RNF182 | RING |
| Q96D59 | RNF183 | RING |
| Q96GF1 | RNF185 | RING |
| Q5TA31 | RNF187 | RING |
| Q6ZMZ0 | RNF19B | RING |
| Q99496 | RNF2 | RING |
| Q5VTR2 | RNF20 | RING |
| Q6ZRF8 | RNF207 | RING |
| Q63HN8 | RNF213 | RING |
| Q8ND24 | RNF214 | RING |
| Q9Y6U7 | RNF215 | RING |
| Q5VTB9 | RNF220 | RING |
| E7ERA6 | RNF223 | RING |
| Q9Y225 | RNF24 | RING |
| Q96BH1 | RNF25 | RING |
| Q9H0A6 | RNF32 | RING |
| Q969K3 | RNF34 | RING |
| Q9H0F5 | RNF38 | RING |
| P78317 | RNF4 | RING |
| O75150 | RNF40 | RING |
| Q68DV7 | RNF43 | RING |
| Q7L0R7 | RNF44 | RING |
| Q99942 | RNF5 | RING |
| Q9Y252 | RNF6 | RING |
| Q9UBF6 | RNF7 | RING |
| O76064 | RNF8 | RING |
| Q5M7Z0 | RNFT1 | RING |
| Q9UNE2 | RPH3AL | RING |
| Q9NSC2 | SALL1 | RING |
| Q9Y467 | SALL2 | RING |
| Q99590 | SCAF11 | RING |
| Q7Z6J0 | SH3RF1 | RING |
| Q149N8 | SHPRH | RING |
| Q8IUQ4 | SIAH1 | RING |
| O43255 | SIAH2 | RING |
| Q96C24 | SYTL4 | RING |
| Q86TM6 | SYVN1 | RING |
| Q9NS56 | TOPORS | RING |
| Q12933 | TRAF2 | RING |
| Q13114 | TRAF3 | RING |
| Q9BUZ4 | TRAF4 | RING |
| O00463 | TRAF5 | RING |
| Q9Y4K3 | TRAF6 | RING |
| Q9BWF2 | TRAIP | RING |
| Q9UDY6 | TRIM10 | TRIM |
| Q96F44 | TRIM11 | TRIM |
| O60858 | TRIM13 | TRIM |
| Q9C019 | TRIM15 | TRIM |
| Q9Y577 | TRIM17 | TRIM |
| Q9C040 | TRIM2 | TRIM |
| P19474 | TRIM21 | TRIM |
| Q8IYM9 | TRIM22 | TRIM |
| P36406 | TRIM23 | TRIM |
| O15164 | TRIM24 | TRIM |
| Q14258 | TRIM25 | TRIM |
| Q12899 | TRIM26 | TRIM |
| P14373 | TRIM27 | TRIM |
| Q13263 | TRIM28 | TRIM |
| O75382 | TRIM3 | TRIM |
| Q9BZY9 | TRIM31 | TRIM |
| Q9UPN9 | TRIM33 | TRIM |
| Q9BYJ4 | TRIM34 | TRIM |
| Q9UPQ4 | TRIM35 | TRIM |
| Q9NQ86 | TRIM36 | TRIM |
| O94972 | TRIM37 | TRIM |
| O00635 | TRIM38 | TRIM |
| Q9HCM9 | TRIM39 | TRIM |
| Q9C037 | TRIM4 | TRIM |
| Q6P9F5 | TRIM40 | TRIM |
| Q8WV44 | TRIM41 | TRIM |
| Q96BQ3 | TRIM43 | TRIM |
| Q9H8W5 | TRIM45 | TRIM |
| Q7Z4K8 | TRIM46 | TRIM |
| Q96LD4 | TRIM47 | TRIM |
| Q8IWZ4 | TRIM48 | TRIM |
| P0CI25 | TRIM49 | TRIM |
| A6NDI0 | TRIM49B | TRIM |
| Q9C035 | TRIM5 | TRIM |
| Q86XT4 | TRIM50 | TRIM |
| Q96A61 | TRIM52 | TRIM |
| Q9BYV2 | TRIM54 | TRIM |
| Q9BRZ2 | TRIM56 | TRIM |
| Q8NG06 | TRIM58 | TRIM |
| P0CI25 | TRIM49 | TRIM |
| Q9C030 | TRIM6 | TRIM |
| Q495X7 | TRIM60 | TRIM |
| Q5EBN2 | TRIM61 | TRIM |
| Q9BVG3 | TRIM62 | TRIM |
| Q969Q1 | TRIM63 | TRIM |
| A6NI03 | TRIM64B | TRIM |
| A6NLI5 | TRIM64C | TRIM |
| Q6PJ69 | TRIM65 | TRIM |
| Q6ZTA4 | TRIM67 | TRIM |
| Q6AZZ1 | TRIM68 | TRIM |
| Q86WT6 | TRIM69 | TRIM |
| Q9C029 | TRIM7 | TRIM |
| Q2Q1W2 | TRIM71 | TRIM |
| Q6ZMU5 | TRIM72 | TRIM |
| Q86UV7 | TRIM73 | TRIM |
| Q86UV6 | TRIM74 | TRIM |
| Q9BZR9 | TRIM8 | TRIM |
| Q9C026 | TRIM9 | TRIM |
| Q8N9V2 | TRIML1 | TRIM |
| Q8TEL6 | TRPC4AP | DCAF |
| P53804 | TTC3 | RING |
| Q96T88 | UHRF1 | RING |
| Q96PU4 | UHRF2 | RING |
| Q9H9P5 | UNKL | RING |
| Q9P253 | VPS18 | RING |
| P49754 | VPS41 | RING |
| Q8N3P4 | VPS8 | RING |
| P98170 | XIAP | RING |
| O95625 | ZBTB11 | RING |
| O60315 | ZEB2 | RING |
| Q9ULJ6 | ZMIZ1 | RING |
| Q8NF64 | ZMIZ2 | RING |
| Q15326 | ZMYND11 | RING |
| Q9ULU4 | ZMYND8 | RING |
| Q9P2Y4 | ZNF219 | RING |
| Q8NCA9 | ZNF784 | RING |
| Q8ND25 | ZNRF1 | RING |
| Q8WWF5 | ZNRF4 | RING |
| Q8NEG5 | ZSWIM2 | RING |
| O43918 | AIRE | SINGLE_OTHER |
| O14965 | AURKA | SINGLE_OTHER |
| Q96GD4 | AURKB | SINGLE_OTHER |
| Q9NXR7 | BABAM2 | SINGLE_OTHER |
| Q9H0C5 | BTBD1 | SINGLE_OTHER |
| Q86UW7 | CADPS2 | SINGLE_OTHER |
| O00257 | CBX4 | SINGLE_OTHER |
| Q9HC52 | CBX8 | SINGLE_OTHER |
| P61024 | CKS1B | SINGLE_OTHER |
| Q92793 | CREBBP | SINGLE_OTHER |
| P02511 | CRYAB | SINGLE_OTHER |
| Q96GG9 | DCUN1D1 | SINGLE_OTHER |
| Q8IWE4 | DCUN1D3 | SINGLE_OTHER |
| Q14241 | ELOA | SINGLE_OTHER |
| Q0VG06 | FAAP100 | SINGLE_OTHER |
| Q9BTP7 | FAAP24 | SINGLE_OTHER |
| O15360 | FANCA | SINGLE_OTHER |
| Q8NB91 | FANCB | SINGLE_OTHER |
| Q00597 | FANCC | SINGLE_OTHER |
| Q9NPI8 | FANCF | SINGLE_OTHER |
| O15287 | FANCG | SINGLE_OTHER |
| Q8IYD8 | FANCM | SINGLE_OTHER |
| Q8NEE6 | FBXL13 | SINGLE_OTHER |
| Q8N461 | FBXL16 | SINGLE_OTHER |
| Q9BSK4 | FEM1A | SINGLE_OTHER |
| Q96JP0 | FEM1C | SINGLE_OTHER |
| P35637 | FUS | SINGLE_OTHER |
| Q8NEA9 | GMCL2 | SINGLE_OTHER |
| P56524 | HDAC4 | SINGLE_OTHER |
| P11142 | HSPA8 | SINGLE_OTHER |
| P51617 | IRAK1 | SINGLE_OTHER |
| Q9NWZ3 | IRAK4 | SINGLE_OTHER |
| Q9H1B7 | IRF2BPL | SINGLE_OTHER |
| Q92830 | KAT2A | SINGLE_OTHER |
| Q92831 | KAT2B | SINGLE_OTHER |
| Q9P0J7 | KCMF1 | SINGLE_OTHER |
| Q9H3F6 | KCTD10 | SINGLE_OTHER |
| Q8NHM5 | KDM2B | SINGLE_OTHER |
| Q96M94 | KLHL15 | SINGLE_OTHER |
| Q9H000 | MKRN2 | SINGLE_OTHER |
| Q13434 | MKRN4P | SINGLE_OTHER |
| Q96JY6 | PDLIM2 | SINGLE_OTHER |
| P78364 | PHC1 | SINGLE_OTHER |
| Q12829 | RAB40B | SINGLE_OTHER |
| Q9UJ41 | RABGEF1 | SINGLE_OTHER |
| P49792 | RANBP2 | SINGLE_OTHER |
| Q96D21 | RASD2 | SINGLE_OTHER |
| Q96G75 | RMND5B | SINGLE_OTHER |
| Q9Y3V2 | RWDD3 | SINGLE_OTHER |
| O43290 | SART1 | SINGLE_OTHER |
| Q9UBV2 | SEL1L | SINGLE_OTHER |
| P52823 | STC1 | SINGLE_OTHER |
| Q8N7C3 | TRIML2 | SINGLE_OTHER |
| Q14669 | TRIP12 | SINGLE_OTHER |
| Q9NRJ4 | TULP4 | SINGLE_OTHER |
| P51668 | UBE2D1 | SINGLE_OTHER |
| Q05086 | UBE3A | SINGLE_OTHER |
| Q15386 | UBE3C | SINGLE_OTHER |
| O94874 | UFL1 | SINGLE_OTHER |
| Q05516 | ZBTB16 | SINGLE_OTHER |
| Q7Z7L7 | ZER1 | SINGLE_OTHER |
| Q6WRX3 | ZYG11A | SINGLE_OTHER |
| Q9C0D3 | ZYG11B | SINGLE_OTHER |
| O60260 | PRKN | RBR |
| Q9BYM8 | RBCK1 | RBR |
| Q7Z419 | RNF144B | RBR |
| Q9NV58 | RNF19A | RBR |
| Q96EP0 | RNF31 | RBR |
| Q9UBS8 | RNF14 | RBR |
| P50876 | RNF144A | RBR |
| Q9P2G1 | ANKIB1 | RBR |
| Q8IWT3 | CUL9 | RBR |
| Q9Y4X5 | ARIH1 | RBR |
| O95376 | ARIH2 | RBR |
| Q9NWF9 | RNF216 | RBR |
| Q9Y314 | NOSIP | UBOX |
| Q13356 | PPIL2 | UBOX |
| Q9UNE7 | STUB1 | UBOX |
| O43734 | TRAF3IP2 | UBOX |
| Q14139 | UBE4A | UBOX |
| O95155 | UBE4B | UBOX |
| O94941 | UBOX5 | UBOX |
| Q8IWV7 | UBR1 | UBOX |
| Q8IWV8 | UBR2 | UBOX |
| Q6ZT12 | UBR3 | UBOX |
| Q5T4S7 | UBR4 | UBOX |
| Q8N806 | UBR7 | UBOX |
| Q8N9V3 | WDSUB1 | UBOX |
| O14682 | ENC1 | BTB_3 |
| Q9H2C0 | GAN | BTB_3 |
| Q9P2D0 | IBTK | BTB_3 |
| Q9Y573 | IPP | BTB_3 |
| C9JR72 | KBTBD13 | BTB_3 |
| Q8IY47 | KBTBD2 | BTB_3 |
| Q8NAB2 | KBTBD3 | BTB_3 |
| Q9NVX7 | KBTBD4 | BTB_3 |
| Q86V97 | KBTBD6 | BTB_3 |
| Q8WVZ9 | KBTBD7 | BTB_3 |
| Q14145 | KEAP1 | BTB_3 |
| Q9NR64 | KLHL1 | BTB_3 |
| Q6JEL2 | KLHL10 | BTB_3 |
| Q9NVR0 | KLHL11 | BTB_3 |
| Q53G59 | KLHL12 | BTB_3 |
| Q9P2N7 | KLHL13 | BTB_3 |
| Q6TDP4 | KLHL17 | BTB_3 |
| O94889 | KLHL18 | BTB_3 |
| Q9Y2M5 | KLHL20 | BTB_3 |
| Q9UJP4 | KLHL21 | BTB_3 |
| Q53GT1 | KLHL22 | BTB_3 |
| Q8NBE8 | KLHL23 | BTB_3 |
| Q6TFL4 | KLHL24 | BTB_3 |
| Q9H0H3 | KLHL25 | BTB_3 |
| Q9NXS3 | KLHL28 | BTB_3 |
| Q96CT2 | KLHL29 | BTB_3 |
| Q9UH77 | KLHL3 | BTB_3 |
| Q96NJ5 | KLHL32 | BTB_3 |
| Q2WGJ6 | KLHL38 | BTB_3 |
| Q96PQ7 | KLHL5 | BTB_3 |
| Q8WZ60 | KLHL6 | BTB_3 |
| Q8IXQ5 | KLHL7 | BTB_3 |
| Q8IXQ5 | KLHL7 | BTB_3 |
| Q9P2J3 | KLHL9 | BTB_3 |
| Q8NDN9 | RCBTB1 | BTB_3 |
| O95199 | RCBTB2 | BTB_3 |
| Q6IQ16 | SPOPL | BTB_3 |
| Q969K4 | ABTB1 | BTB_OTHER |
| Q8N961 | ABTB2 | BTB_OTHER |
| Q8N143 | BCL6B | BTB_OTHER |
| Q96Q07 | BTBD9 | BTB_OTHER |
| Q8NFY9 | KBTBD8 | BTB_OTHER |
| Q693B1 | KCTD11 | BTB_OTHER |
| Q8WZ19 | KCTD13 | BTB_OTHER |
| Q4G0X4 | KCTD21 | BTB_OTHER |
| Q9NXV2 | KCTD5 | BTB_OTHER |
| Q8NC69 | KCTD6 | BTB_OTHER |
| Q8N4N3 | KLHL36 | BTB_OTHER |
| Q8N653 | LZTR1 | BTB_OTHER |
| Q96RE7 | NACC1 | BTB_OTHER |
| O94844 | RHOBTB1 | BTB_OTHER |
| Q13829 | TNFAIP1 | BTB_OTHER |
| Q86UZ6 | ZBTB46 | BTB_OTHER |
| Q96BR9 | ZBTB8A | BTB_OTHER |
| Q12834 | CDC20 | CDC20 |
| Q9UM11 | FZR1 | CDC20 |
| Q13685 | AAMP | DWD |
| Q9C0C7 | AMBRA1 | DCAF |
| O14727 | APAF1 | DWD |
| Q676U5 | ATG16L1 | DWD |
| Q9NSI6 | BRWD1 | DCAF |
| O43684 | BUB3 | DWD |
| O76071 | CIAO1 | DWD |
| Q8NHY2 | COP1 | DCAF |
| Q05048 | CSTF1 | DWD |
| Q96SW2 | CRBN | DCAF |
| Q9Y4B6 | DCAF1 | DCAF |
| Q5QP82 | DCAF10 | DCAF |
| Q8TEB1 | DCAF11 | DCAF |
| Q5T6F0 | DCAF12 | DCAF |
| Q9NV06 | DCAF13 | DCAF |
| Q8WV16 | DCAF4 | DCAF |
| Q96JK2 | DCAF5 | DCAF |
| Q58WW2 | DCAF6 | DCAF |
| P61962 | DCAF7 | DCAF |
| Q5TAQ9 | DCAF8 | DCAF |
| Q66K64 | DCAF15 | DCAF |
| Q9NXF7 | DCAF16 | DCAF |
| Q92466 | DDB2 | DCAF |
| Q9BW61 | DDA1 | DCAF |
| Q5H9S7 | DCAF17 | DCAF |
| Q8NFQ8 | IFRG15 | DCAF |
| Q9BTV6 | DPH7 | DWD |
| Q9NZJ0 | DTL | DCAF |
| O75530 | EED | DWD |
| Q13347 | EIF31 | DWD |
| Q53HC9 | EIPR1 | DWD |
| Q05BV3 | EML5 | DWD |
| Q6ZMW3 | EML6 | DWD |
| Q13216 | ERCC8 | DCAF |
| Q8TEQ6 | GEMIN5 | DWD |
| P62873 | GNB1 | DWD |
| P62879 | GNB2 | DWD |
| P16520 | GNB3 | DWD |
| O14775 | GNB5 | DWD |
| Q9BQ67 | GRWD1 | DWD |
| Q8WUA4 | GTF3C2 | DWD |
| Q9BVA0 | KATNB1 | DWD |
| Q9UFC0 | LRWD1 | DWD |
| Q9BVC4 | MLST8 | DWD |
| Q9NVX2 | NLE1 | DWD |
| Q8NFH3 | NUP43 | DWD |
| Q9BRP4 | PAAF1 | DWD |
| P43034 | PAFAH1B1 | DWD |
| O00628 | PEX7 | DWD |
| Q8WWQ0 | PHIP | DCAF |
| Q9Y263 | PLAA | DWD |
| O43660 | PLRG1 | DWD |
| Q8NBT0 | POC1A | DWD |
| Q8TC44 | POC1B | DWD |
| Q9UMS4 | PRPF19 | DWD |
| O43172 | PRPF4 | DWD |
| Q13610 | PWP1 | DWD |
| Q15269 | PWP2 | DWD |
| P63244 | RACK1 | DWD |
| P78406 | RAE1 | DWD |
| Q09028 | RBBP4 | DWD |
| Q15291 | RBBP5 | DWD |
| Q16576 | RBBP7 | DWD |
| P55735 | SEC13 | DWD |
| Q9NQW1 | SEC31B | DWD |
| Q96EE3 | SEH1L | DWD |
| Q2TAY7 | SMU1 | DWD |
| Q96DI7 | SNRNP40 | DWD |
| Q9NRL3 | STRN4 | DWD |
| Q15542 | TAF5 | DWD |
| O75529 | TAF5L | DWD |
| Q9BQ87 | TBL1Y | DWD |
| Q12788 | TBL3 | DWD |
| Q99973 | TEP1 | DWD |
| Q96J01 | THOC3 | DWD |
| Q86W42 | THOC6 | DWD |
| Q04724 | TLE1 | DWD |
| Q04725 | TLE2 | DWD |
| Q04726 | TLE3 | DWD |
| Q6Q0C0 | TRAF7 | DWD |
| O75717 | WDHD1 | DWD |
| O75083 | WDR1 | DWD |
| Q9GZL7 | WDR12 | DWD |
| Q8IZU2 | WDR17 | DWD |
| Q9BV38 | WDR18 | DWD |
| Q9H7D7 | WDR26 | DWD |
| Q9UNX4 | WDR3 | DWD |
| Q9C0J8 | WDR33 | DWD |
| Q9Y218 | WDR37 | DWD |
| Q9HAD4 | WDR41 | DWD |
| O94967 | WDR47 | DWD |
| Q8TAF3 | WDR48 | DWD |
| P61964 | WDR5 | DWD |
| Q7Z5U6 | WDR53 | DWD |
| Q6PJI9 | WDR59 | DWD |
| Q86VZ2 | WDR5B | DWD |
| Q9NNW5 | WDR6 | DWD |
| Q9GZS3 | WDR61 | DWD |
| Q9H967 | WDR76 | DWD |
| Q9BQA1 | WDR77 | DWD |
| Q6UXN9 | WDR82 | DWD |
| Q6ZQQ6 | WDR87 | DWD |
| Q6ZMY6 | WDR88 | DWD |
| Q96FK6 | WDR89 | DWD |
| Q96MX6 | DNAAF10 | DWD |
| Q8N5D0 | WDTC1 | DCAF |
| Q9Y617 | WSB1 | DWD |
| Q9Y297 | BTRC | F-Box |
| P41002 | CCNF | F-Box |
| Q99618 | CDCA3 | F-Box |
| Q008S8 | ECT2L | F-Box |
| Q8NFZO | FBH1 | F-Box |
| Q9NXK8 | FBXL12 | F-Box |
| Q8N1E6 | FBXL14 | F-Box |
| Q9H469 | FBXL15 | F-Box |
| Q9UF56 | FBXL17 | F-Box |
| Q96ME1 | FBXL18 | F-Box |
| Q6PCT2 | FBXL19 | F-Box |
| Q9UKC9 | FBXL2 | F-Box |
| Q96IG2 | FBXL20 | F-Box |
| Q9UKT6 | FBXL21P | F-Box |
| Q6P050 | FBXL22 | F-Box |
| Q9UKT7 | FBXL3 | F-Box |
| Q9UKA2 | FBXL4 | F-Box |
| Q9UKA1 | FBXL5 | F-Box |
| Q8N531 | FBXL6 | F-Box |
| Q9UJT9 | FBXL7 | F-Box |
| Q96CD0 | FBXL8 | F-Box |
| Q9UK96 | FBXO10 | F-Box |
| Q86XK2 | FBXO11 | F-Box |
| Q8NCQ5 | FBXO15 | F-Box |
| Q8IX29 | FBXO16 | F-Box |
| Q96EF6 | FBXO17 | F-Box |
| Q8NEZ5 | FBXO22 | F-Box |
| O75426 | FBXO24 | F-Box |
| Q8TCJ0 | FBXO25 | F-Box |
| Q8NI29 | FBXO27 | F-Box |
| Q9NVF7 | FBXO28 | F-Box |
| Q9UK99 | FBXO3 | F-Box |
| Q8TB52 | FBXO30 | F-Box |
| Q5XUX0 | FBXO31 | F-Box |
| Q969P5 | FBXO32 | F-Box |
| Q7Z6M2 | FBXO33 | F-Box |
| Q9NWN3 | FBXO34 | F-Box |
| Q8NEA4 | FBXO36 | F-Box |
| Q8N4B4 | FBXO39 | F-Box |
| Q9UKT5 | FBXO4 | F-Box |
| Q9UH90 | FBXO40 | F-Box |
| Q8TF61 | FBXO41 | F-Box |
| Q6P3S6 | FBXO42 | F-Box |
| Q9H4M3 | FBXO44 | F-Box |
| P0C2W1 | FBXO45 | F-Box |
| Q6PJ61 | FBXO46 | F-Box |
| Q5MNV8 | FBXO47 | F-Box |
| Q5FWF7 | FBXO48 | F-Box |
| Q9UKT4 | FBXO5 | F-Box |
| Q9NRD1 | FBXO6 | F-Box |
| Q9Y311 | FBXO7 | F-Box |
| Q9NRD0 | FBXO8 | F-Box |
| Q9UK97 | FBXO9 | F-Box |
| Q5XX13 | FBXW10 | F-Box |
| Q9UKB1 | FBXW11 | F-Box |
| Q6X9E4 | FBXW12 | F-Box |
| Q9UKT8 | FBXW2 | F-Box |
| P57775 | FBXW4 | F-Box |
| Q969U6 | FBXW5 | F-Box |
| Q969H0 | FBXW7 | F-Box |
| Q8N3Y1 | FBXW8 | F-Box |
| Q5XUX1 | FBXW9 | F-Box |
| Q9Y2K7 | KDM2A | F-Box |
| Q13309 | SKP2 | F-Box |
| O60907 | TBL1X | F-Box |
| Q9BZK7 | TBL1XR1 | F-Box |
| Q86WB0 | ZC3HC1 | F-Box |
| Q9Y576 | ASB1 | SOCS_VHL_BC-BOX |
| Q8WX13 | ASB10 | SOCS_VHL_BC-BOX |
| Q8WXH4 | ASB11 | SOCS_VHL_BC-BOX |
| Q8WXK4 | ASB12 | SOCS_VHL_BC-BOX |
| Q8WXK3 | ASB13 | SOCS_VHL_BC-BOX |
| Q96NS5 | ASB16 | SOCS_VHL_BC-BOX |
| Q8WXJ9 | ASB17 | SOCS_VHL_BC-BOX |
| Q96Q27 | ASB2 | SOCS_VHL_BC-BOX |
| Q9Y575 | ASB3 | SOCS_VHL_BC-BOX |
| Q9Y574 | ASB4 | SOCS_VHL_BC-BOX |
| Q8WWX0 | ASB5 | SOCS_VHL_BC-BOX |
| Q9NWX5 | ASB6 | SOCS_VHL_BC-BOX |
| Q9H672 | ASB7 | SOCS_VHL_BC-BOX |
| Q9H765 | ASB8 | SOCS_VHL_BC-BOX |
| Q96DX5 | ASB9 | SOCS_VHL_BC-BOX |
| Q9NSE2 | CISH | SOCS_VHL_BC-BOX |
| P57737 | CORO7 | SOCS_VHL_BC-BOX |
| Q9UK73 | FEM1B | SOCS_VHL_BC-BOX |
| Q96L50 | LRR1 | SOCS_VHL_BC-BOX |
| Q15345 | LRRC41 | SOCS_VHL_BC-BOX |
| Q96G25 | MED8 | SOCS_VHL_BC-BOX |
| Q96EF0 | MTMR8 | SOCS_VHL_BC-BOX |
| Q9BR09 | NEURL2 | SOCS_VHL_BC-BOX |
| Q96MG8 | PCMTD1 | SOCS_VHL_BC-BOX |
| Q8WXH6 | RAB40A | SOCS_VHL_BC-BOX |
| P0C0E4 | RAB40AL | SOCS_VHL_BC-BOX |
| Q96S21 | RAB40C | SOCS_VHL_BC-BOX |
| O15524 | SOCS1 | SOCS_VHL_BC-BOX |
| O14543 | SOCS3 | SOCS_VHL_BC-BOX |
| Q8WXH5 | SOCS4 | SOCS_VHL_BC-BOX |
| O75159 | SOCS5 | SOCS_VHL_BC-BOX |
| O14544 | SOCS6 | SOCS_VHL_BC-BOX |
| O14512 | SOCS7 | SOCS_VHL_BC-BOX |
| Q96BD6 | SPSB1 | SOCS_VHL_BC-BOX |
| Q99619 | SPSB2 | SOCS_VHL_BC-BOX |
| Q6PJ21 | SPSB3 | SOCS_VHL_BC-BOX |
| Q96A44 | SPSB4 | SOCS_VHL_BC-BOX |
| P82094 | TMF1 | SOCS_VHL_BC-BOX |
| P40337 | VHL | SOCS_VHL_BC-BOX |
| Q9NYS7 | WSB2 | SOCS_VHL_BC-BOX |

In some cases, the E3 ligase substrate receptor protein is at least 80%, e.g., at least 90%, at least 95%, or at least 99% identical to an E3 ligase substrate receptor protein from Table 1.

In some cases, the E3 ligase is an enzymatically active portion of an E3 ligase substrate receptor protein from Table 1.

### E3 Ligase Binding Modulators

The methods described herein are useful, for example, for identifying programmable E3 ligase substrate receptor proteins, optionally along with their neosubstrates. In some cases, the methods are used to validate and/or identify E3 ligase substrate receptor proteins that selectively interact with target proteins (e.g., neosubstrates), in the presence of a compound, e.g., an E3 ligase binding modulator such as a molecular glue, e.g., a molecular glue degrader.

E3 ligase binding modulators, e.g., cereblon binding modulators, are described, for example, in WO2021/069705, WO2021/053555, WO2022/152821, WO2022/219407, and WO2022219412, which are hereby incorporated by reference in their entirety, as well as in PCT/US2022/050242, which is hereby incorporated by reference in its entirety and which is attached hereto as Appendix A. In some cases, the E3 ligase binding modulator, e.g., cereblon binding modulator, is a compound described in any of WO2021/069705, WO2021/053555, WO2022/152821, WO2022/219407, and WO2022219412, or PCT/US2022/050242, or a pharmaceutically acceptable salt thereof, or a stereoisomer thereof.

### Molecular Glues

In some cases, the E3 ligase binding modulator is a molecular glue.

A molecular glue is a small molecule that stabilizes the interaction of two or more biomolecules (e.g., proteins) at a protein-protein interaction (PPI) interface, e.g., by chemically inducing or strengthening surface interactions between the proteins. In some cases, the molecular glue stabilizes the interaction of an E3 ligase substrate receptor protein and one or more target protein(s).

In some cases, the molecular glue functions as a molecular glue drug by modulating (e.g., increasing or promoting) one or more of: the stability of protein-protein interaction(s), degradation of protein(s), sequestration of protein(s) (e.g., into specific regions of a cell), phosphorylation of protein(s), de-phosphorylation of protein(s), and stabilization of protein(s).

In some cases, the modulation is directly of the target protein (the “glued” target). In some cases, the modulation is indirect (e.g., of a target downstream of the “glued” target).

### Molecular Glue Degraders

Thalidomide and immunomodulatory imide drugs (IMiDs), such as lenalidomide, and pomalidomide, are examples of molecular glue drugs that induce degradation of normally unrecognized target proteins (sometimes referred to as “neosubstrates”) by generating an interaction between an E3 ligase substrate receptor (e.g., cereblon) and a target protein (e.g., IKZF1/3).

Molecular glue drugs, such as these, that induce the degradation of protein(s) are sometimes referred to as a molecular glue degraders. Molecular glue degraders are believed to create neosubstrate recognition interfaces on the surface of the E3 ligase substrate receptor protein that engage in induced protein-protein interactions with neosubstrates.

### Target Proteins

The compositions and methods describe herein are useful, for example, in identification and/or prediction of programmable E3 ligase substrate receptor proteins and, optionally, target proteins, e.g., as described in PCT/US2022/050242, which is hereby incorporated by reference in its entirety, and which is attached hereto as Appendix A.

### Structural Features

The methods described herein utilize structural features of proteins. Any of the structural features described herein can be utilized alone or in combination with any of the other structural features described herein.

In some cases, the structural features comprise molecular surface features.

In some cases, the structural features comprise geometric, chemical, and/or evolutionary features. See, e.g., Tubiana et al., “ScanNet: an interpretable geometric deep learning model for structure-based protein binding site prediction,” Nature Methods 19:730-39 (2022), which is hereby incorporated by reference in its entirety. In some cases, the structural features comprise one or more of: amino acid type (e.g., one-hot encoded, 20 dimensions), secondary structure type (e.g., one-hot encoded, eight dimensions, e.g., computed with DSSP61), relative accessible surface area (e.g., one dimension, e.g., computed with DSSP61), coordination number (e.g., one dimension, e.g., defined as the number of Ca atoms in a ball of radius 13 center around the Ca atom of the amino acid), half-sphere exposure index82 (e.g., one dimension, e.g., defined as follows: let N1 be the coordination number, and N2 the number of Cα atoms in the intersection of a ball of radius 13 center and above the plane defined by the Cα−Cβ vector. The half-sphere exposure index is 2N2−N1N1∈[−1,1]), backbone and sidechain depth83 (e.g., two dimensions, e.g., molecular surface computed using MSMS (probe radius 1.5 Å) 84, and the distance to the surface computed and averaged for all backbone (resp. sidechain) atoms), surface convexity index (e.g., three dimensions, e.g., for each atom, a ball of radius 5, 8 or 11 Å centered on it is computed, and compute the f fraction of its volume located on the inside of molecular surface; the index is given by 2f−1∈(−1,−1); the surface convexity index is averaged at the amino acid level), position-weight matrix (e.g., 21 dimensions), conservation score C=log 21+Σa log PWM(a) (e.g., one dimension).

In some cases, the structural features comprise atomic elements(s). See, e.g., Krapp et. al., “PeSTo: parameter-free geometric deep learning for accurate prediction of protein binding interfaces,” Nature Communications 14:2175 (2023), which is hereby incorporated by reference in its entirety.

### Molecular Surface Features

The molecular surface is a higher-level representation of protein structure than protein structure or sequence. It models a protein as a continuous shape with geometric and chemical features. See Richards et al., “Ann. Rev. Biophysics Bioeng. 6:151-76 (2003).

The molecular surface is useful for the methods described herein, for example, for identifying proteins with similar and/or complementary surface features, predicting molecular interactions between an E3 ligase and a target protein and/or binding modulator. Thus, in some cases, the methods described herein comprise providing molecular surface feature(s) of one or more protein(s). Molecular surface features that are useful for the methods described herein include, for example, geometric features and/or chemical features.

In some cases, the molecular surface features are extracted from a crystal structure. In some cases, the crystal structure is a ligand bound (i.e. holo). In some cases, the crystal structure is unbound (i.e. apo). In some cases, the molecular surface features are extracted from a computer modeled structure. In some cases, the computer modeled structure is ligand bound. In some cases, the computer modeled structure is unbound.

In some cases, the molecular surface features are obtained from a database. For example, the Protein Data Bank (PDB, rcsb.org) or the AlphaFold Protein Structure Database (alphafold.ebi.ac.uk).

PDB is a database for the three-dimensional structural data of large biological molecules, such as proteins and nucleic acids (Nucleic Acids Res. 2019 Jan. 8; 47(D1):D520-D528. doi: 10.1093/nar/gky949). The data is submitted by biologists and biochemists from around the world, are freely accessible on the Internet via the websites of its member organizations (e.g. PDBe—pdbe.org, PDBj—pdbj.org, RCSB—rcsb.org/pdb, and BMRB—bmrb.wisc.edu). The PDB is overseen by an organization called the Worldwide Protein Data Bank—wwPDB—.

In some embodiments, providing molecular surface feature(s) comprises determining a three-dimensional structure experimentally, e.g., using X-ray crystallography, nuclear magnetic resonance (NMR spectroscopy), cryo-electron microscopy (cryoEM), small-angle X-ray scattering (SAXS), small-angle neutron scattering (SANS), or combinations thereof.

In some embodiments, providing molecular surface feature(s) comprises modeling of the three-dimensional structural context, e.g., if the three-dimensional structure of the identified protein is not known.

In some cases, modeling of the three-dimensional structural context is carried out using computer modeling. In some cases, the computer modeling is carried out using an artificial intelligence model, e.g., according to the methods described in Jumper et al., “Highly Accurate Protein Structure Prediction with AlphaFold,” Nature 596:583-89 (2021) or Evans et al., “Protein Complex Prediction with AlphaFold-Multimer,” bioRxiv doi.org/10.1101/2021.10.04.463034 (2021).

The molecular surface feature(s) can be provided together or separately. In some cases, the structure of one or more of the proteins is a ligand bound (i.e. holo) structure. In some cases, the structure of one or more of the proteins is unbound (i.e. apo).

In some cases, the molecular surface features(s) are based on the three-dimensional structure of a region of a protein, e.g., the interface region of the protein that participates in (or is hypothesized to participate in) a PPI.

In some cases, for example, where the three-dimensional structures are unbound, starting structure(s) are built by superimposing the three-dimensional structures onto a reference structure.

In some cases, the molecular surface feature(s) are provided as parameters in digital format, e.g., in a MasIF data file, for use in the methods described herein. Thus, in some cases, the methods described herein comprise providing data defining the molecular surface feature(s) of two or more proteins (or fragments thereof).

Molecular surface features defining protein surface patches, as well as methods for obtaining data defining such surface patches, are further described in PCT/US2022/050275, which is hereby incorporated by reference in its entirety, and which is attached as Appendix B.

In some cases, the molecular surface feature(s) are geometric feature(s) and/or chemical feature(s).

### Geometric Features

In some cases, the surface feature(s) are geometric feature(s). In some cases, the geometric feature(s) are selected from the group consisting of a shape index (Koenderink et al., “Surface Shape and Curvature Scales,” Image Vis. Comput. 10:557-64 (1992), which is hereby incorporated by reference in its entirety), distance-dependent curvature (Yin et al., “Fast Screening of Protein Surfaces using Geometric Invariant Fingerprints” Proc. Natl. Acad. Sci. USA 106:16622-26 (2009), which is hereby incorporated by reference in its entirety), geodesic polar coordinate(s), radial (angular) coordinate(s), and combinations thereof. In other cases, the geometric features are learned directly from the underlying tertiary structure of the protein and its atomic arrangements.

### Chemical Features

In some cases, the surface feature(s) are chemical feature(s). In some cases, the chemical feature(s) are selected from the group consisting of hydropathy index (Kyte et al., “A Simple Method for Displaying the Hydropathic Character of a Protein” J. Mol. Biol. 157:105-32 (1982), which is hereby incorporated by reference in its entirety), continuum electrostatics (Jurrus et al. “Improvements to the APBS Biomolecular Solvation Software Suite,” Protein Sci. 27:112-28 (2018), which is hereby incorporated by reference in its entirety), location of free electrons (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), location of free proton donors (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), and combinations thereof. In other cases, the chemical feature(s) are learned directly from the underlying tertiary structure of the protein and its atomic arrangements.

Identification and Characterization of E3 Ligase Substrate Receptor Proteins and/or their Interaction Partners

Provided herein are compositions and methods for identification, classification, and/or selection of programmable E3 ligase substrate receptor protein(s), and, optionally, their interaction partners (e.g., corresponding neosubstrate(s)).

In some cases, the method is performed at least in part by one or more computers; and one or more storage devices communicatively coupled to the one or more computers, wherein the one or more storage devices store instructions that, when executed by the one or more computers, cause the one or more computers to perform operations comprising the methods described herein. Such systems are provided herein. Also provided herein are one or more non-transitory computer storage media storing instructions that when executed by one or more computers cause the one or more computers to perform operations comprising the method of any one of the preceding claims.

In some cases, the methods described herein comprise providing a set of structural features (e.g., molecular surface features), e.g., as described herein, of one or more protein(s). In some cases, the structural features describe a protein surface. In some cases, the structural features describe a space complementary to a protein surface.

In some cases, the methods described herein comprise providing a set of structural features (e.g., molecular surface features), e.g., as described herein of E3 ligase substrate receptor protein(s). In some cases, the structural features of the E3 ligase substrate receptor protein is in an unbound state (e.g., an E3 ligase “surface”). In some cases, the structural features of the E3 ligase substrate receptor protein is in a bound state (e.g., an E3 ligase “neosurface”).

In some cases, the methods are carried out using a pipeline that exploits geometric deep learning to process the structural feature data which lies in a non-Euclidean domain.

In some cases, classifying an E3 ligase substrate receptor protein as programmable comprises comparing a programmability score for the E3 ligase substrate receptor protein to a threshold and, if a) the threshold is satisfied and b) optionally if a cysteine is present, classifying the E3 ligase substrate receptor protein as programmable; else, classifying the E3 ligase substrate receptor protein as not programmable. In some cases, the threshold is a programmability score for a known E3 ligase substrate receptor protein, optionally CRBN.

In some cases, classifying an E3 ligase substrate receptor protein as desirable comprises comparing a desirability score for the E3 ligase substrate receptor protein to a threshold criterion and, if a) the threshold criterion is satisfied, classifying the E3 ligase substrate receptor protein as desirable; else, classifying the E3 ligase substrate receptor protein as not desirable. In some cases, the threshold is a desirability score for a known E3 ligase substrate receptor protein, optionally CRBN.

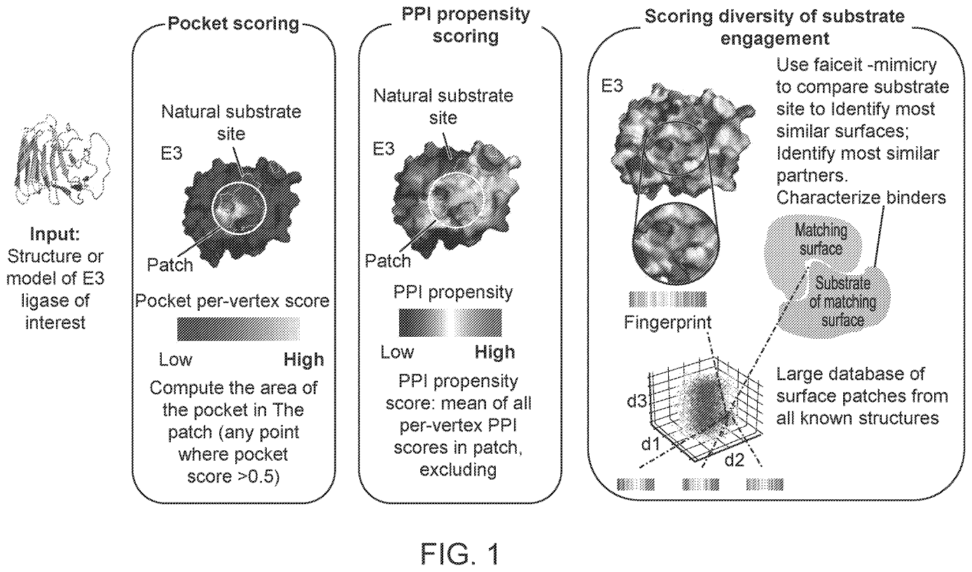

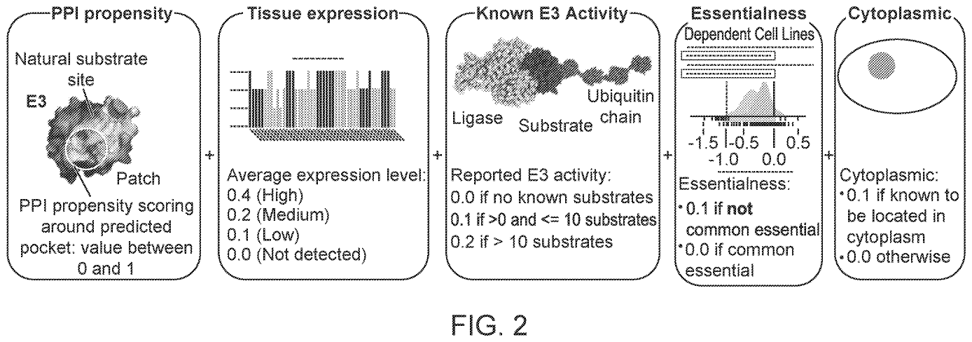

Examples workflows of methods for identification and characterization of E3 ligase substrate receptor proteins are shown, for example, in FIG. 1 and FIG. 2.

### Programmability Score

In some cases, the methods described herein comprise calculating a programmability score, e.g., for a known or suspected E3 ligase substrate receptor protein. In some cases, the programmability score is calculated based on a PPI propensity score and/or a ligandability score, e.g., as described herein. In some cases, the programmability score is: 0.5*ligandability score+0.5*PPI propensity score.

In some cases, the methods described herein comprise a method for calculating a programmability score for a known or suspected E3 ligase substrate receptor protein, comprising: a) providing data comprising a first set of structural features, optionally molecular surface features representing a putative interaction region on a protein known or suspected to be an E3 ligase substrate receptor protein, optionally a protein listed in Table 1; and, based on the first set of structural features, calculating a PPI propensity score for the protein, optionally using a machine learning algorithm trained on structural features of known or predicted protein-protein interaction surfaces; b) providing data comprising a second set of structural features, optionally molecular surface features representing a putative pocket region on the protein, and, based on the second set of structural features, calculating a ligandability score for the protein, optionally using a machine learning algorithm trained on structural features of known or predicted high affinity small molecule pockets; c) based on the PPI propensity score and the ligandability score, calculating a programmability score for the known or suspected E3 ligase substrate receptor protein; d) optionally identifying the presence or absence of a cysteine residue within the putative pocket region; and e) optionally, classifying the E3 ligase substrate receptor protein as programmable or not based on the programmability score and optionally the presence or absence of a cysteine residue within the putative pocket region.

### Ligandability Score

In some cases, calculating a ligandability score is based on a pocket prediction algorithm, e.g., as described in Le Guilloux, Vincent, Peter Schmidtke, and Pierre Tuffery. “Fpocket: an open source platform for ligand pocket detection.” BMC bioinformatics 10.1 (2009): 1-11, Roy, Ambrish, Jianyi Yang, and Yang Zhang. “COFACTOR: an accurate comparative algorithm for structure-based protein function annotation.” Nucleic acids research 40.W1 (2012): W471-W477, Ravindranath, Pradeep Anand, and Michel F. Sanner. “AutoSite: an automated approach for pseudo-ligands prediction—from ligand-binding sites identification to predicting key ligand atoms.” Bioinformatics 32.20 (2016): 3142-3149, Jiménez, José, et al. “DeepSite: protein-binding site predictor using 3D-convolutional neural networks.” Bioinformatics 33.19 (2017): 3036-3042, or Mylonas, Stelios K., Apostolos Axenopoulos, and Petros Daras. “DeepSurf: a surface-based deep learning approach for the prediction of ligand binding sites on proteins.” Bioinformatics 37.12 (2021): 1681-1690, each of which is hereby incorporated by reference in its entirety.

In some cases, calculating a ligandability score comprises training and implementing a machine learning based algorithm, e.g., a neural network, e.g., a 3D (three-dimensional) convolutional neural network, graph neural network, or point-set network

In some cases, calculating a ligandability score comprises: generating an embedding for the putative pocket region by processing the data comprising the first set of structural features using an embedding neural network to generate an embedding of the putative pocket region in an embedding space; and determining, for the putative pocket region, the likelihood of being a ligand binding pocket.

In some cases, calculating a ligandability score comprises assigning a per-surface-vertex regression score (ranging from 0 to 1) to every vertex on the surface of the protein. In this case, a 1 means a high confidence that the vertex is part of a pocket, while a 0 means that there is low confidence the pocket belongs to a pocket. In some cases, a radial patch, e.g., of radius 12 angstroms, is identified in the protein as a pocket containing patch, e.g., using any one of the following criteria: averaging over all the per-vertex pocket scores for each patch in the protein and selecting the top one; averaging over all the per-vertex pocket scores for each patch in the protein and selecting the top one that also contains an exposed cysteine; averaging over all the per-vertex pocket scores for each patch in the protein and selecting the top one that is adjacent to a known substrate site of the E3 ligase. In some cases, once a pocket patch is identified, the ligandability score for that pocket is the average for all the points in the patch.

### PPI Propensity Score

In some cases, calculating a PPI propensity score comprises assigning a per-surface-vertex regression score (ranging from 0 to 1) to every vertex on the surface of the protein. In this case, a 1 indicates a high propensity to form an interaction while a 0 indicates a low propensity to form an interaction.

In some cases, calculating a PPI propensity score is based on a one-body prediction of protein interactions, e.g., as described in Gainza et al., “Deciphering interaction fingerprints from protein molecular surfaces using geometric deep learning,” Nature Methods 17:184-92 (2020), Tubiana et al., “ScanNet: an interpretable geometric deep learning model for structure-based protein binding site prediction,” Nature Methods 19:730-39 (2022), or Krapp et. al., “PeSTo: parameter-free geometric deep learning for accurate prediction of protein binding interfaces,” Nature Communications 14:2175 (2023), each of which is hereby incorporated by reference in its entirety.

In some cases, calculating a PPI propensity score comprises generating an embedding for the putative interaction region by processing the data comprising a first set of structural features using an embedding neural network to generate an embedding of the putative interaction region in an embedding space; and determining, for the putative interaction region, the likelihood of being involved in a protein-protein interaction.

In some cases, all patches adjacent to a pocket patch (e.g., identified as described above, e.g., whose center vertex is within 12 angstroms of the center vertex of the pocket) are evaluated for their PPI propensity, e.g., by computing mean PPI propensity score, but excluding any vertices where the pocket score is greater than a threshold value, e.g., 0.5.

### Desirability Score

In some cases, the methods described herein comprise calculating a desirability score, e.g., for a known or suspected E3 ligase substrate receptor protein. In some cases, the desirability score is calculated based on one or more of: an activity score, a PPI propensity score, essentialness score, expression score, cytoplasm score, and ligandability score (e.g., as described herein).

In some cases, the desirability score is a summation of the activity score, PPI propensity score, cytoplasm score, expression score, and essentialness score. In particular,

$$Desirability Score = Activity Score + PPI Propensity Score + Cytoplasm Score + Expression Score + Essentialness Score$$

In some cases, the desirability score is normalized (e.g. to the maximum desirability score, e.g., by dividing by the maximum desirability score). In some cases, the maximum desirability score is the maximum score in a set of known or suspected E3 ligase substrate receptor proteins. In some cases, the maximum desirability score is the maximum possible score of a theoretical protein.

For example, for a theoretical protein, in the case where the maximum activity score is 0.2, the maximum PPI propensity score is 1, the maximum essentialness score is 0.1, the maximum expression score is 0.4, the maximum cytoplasm score is 0.1, and the maximum ligandablity score is 1, the maximum desirability score would be: 0.2+1+0.1+0.4+0.1+1=2.8, and the normalized desirability score for a known or suspected E3 ligase substrate receptor protein would be X/2.8, where X is the non-normalized desirability score.

In one example, the Desirability Score for each of 634 known or suspected E3 ligase substrate receptor proteins was calculated as described herein. The maximum desirability score observed for the 634 proteins was 1.4725. Included among the 634 was CRBN, which had a desirability score of 1.335. The normalized desirability score (based on the maximum observed) for CRBN was 1.335/1.4725=0.907.

### Activity Score

In some cases, calculating an activity score comprises assigning an activity value to a known or suspected E3 ligase substrate receptor protein based on the number of known interactions between the known or suspected E3 ligase substrate receptor protein and substrate(s).

In some cases, the number of known interactions is reported in the literature (e.g., UbiBrowser (Wang et al., “UbiBrowser 2.0: a comprehensive resource for proteome-wide ubiquitin ligase/deubiquitinase-substrate interactions in eukaryotic species,” Nucleic Acids Research 50: D719-D728 (2022), e.g., available at ubibrowser.bio-it.cn/ubibrowser_v3/Public/download/literature/literature.E3.txt).

Assigning the activity value comprises, for example, binning the count of known interactions of a set of known or suspected E3 ligase substrate receptor proteins and assigning a normalized count value to a known or suspected E3 ligase substrate receptor protein based on the binned counts. For example, in some cases, normalized count values are assigned as follows:

| Normalized Count Value | Count of Known Interactions |
|---|---|
| 0.0 | 0 |
| 0.1 | >0 and <=10 |
| 0.2 | >10 |

Other normalized count values (e.g., between 0.1 and 0.5) are contemplated, e.g., to correspond to other binned counts of known interactions (e.g., 0, >0 and <=X, and >X). Additional granularity (e.g., any number from 2 to 20 bins) in assigning an activity value is also contemplated.

### PPI Propensity Score

In some cases, calculating a PPI propensity score (i.e., between 0 and 1) is carried out as described above.

### Essentialness Score

In some cases, calculating an essentialness score comprises classifying the known or suspected E3 ligase substrate receptor protein as “Essential” or not and, if “Essential,” assigning an essentialness score of 0 and, if not, assigning an essentialness score >0 (e.g., 1). Optionally, an additional penalty is applied (e.g., by multiplying the essentialness score by a factor, e.g., of <1, e.g., from 0 to 0.3, optionally 0.1.

Classifying the known or suspected E3 ligase substrate receptor protein as “Essential” is based, for example, on the DepMap (depmap.org/portal/) annotation of “Common Essential,” e.g., at depmap.org/portal/api/download/gene_dep_summary (see also Tsherniak et al., “Defining a cancer dependency map,” Cell, 170 (3), 564-576 (2017). For example, if “Common Essential,” the unpenalized essentialness score is 0 and if not, the unpenalized essentialness score is 1; with the optional penalty score (e.g., of X) applied, the essentialness score would be 0 if “Common Essential” (0*X) or X if not (1*X); with X=0.1, for example, the essentialness score would be 0 or 0.1.

### Expression Score

In some cases, calculating an expression score comprises assigning an expression value to a known or suspected E3 ligase substrate receptor protein based on the amount of known expression of the known or suspected E3 ligase substrate receptor protein. In some cases, the amount of known expression is the average expression across all tissues; in some cases, the amount of known expression is the tissue expression in a tissue or tissue(s) of interest (e.g., a therapeutic target).

In some cases, the amount of known expression is reported in the literature, e.g., the Human Protein Atlas (see Uhlen et al., “Tissue-based map of the human proteome,” Science 347 (6220): 1260419 (2015) (www.proteinatlas.org), e.g., at www.proteinatlas.org/download/normal tissue.tsv.zip.

Assigning the expression value comprises, for example, assigning a numeric expression value to a class or range of expression levels. For example, in some cases, numeric values are assigned to an expression class (e.g., from the average expression on all tissues, as reported by the Human Protein Atlas), as follows:

| Numeric Expression Value | Expression Class |
|---|---|
| 0.4 | High |
| 0.2 | Medium |
| 0.1 | Low |
| 0 | Not detected |

Other Numeric Expression Values (e.g., between 0 and 1) are contemplated, e.g., corresponding to other expression classes (e.g., different categorical descriptions of expression level).

### Cytoplasm Score

In some cases, calculating a cytoplasm score comprises assigning a cytoplasm value to a known or suspected E3 ligase substrate receptor protein based on whether or not the protein is known to be found within the cytoplasm. In some cases, the cytoplasm value is 0 if the protein is not known to be found within the cytoplasm and 1 if the protein is known to be found within the cytoplasm. Optionally, an additional penalty is applied (e.g., by multiplying the cytoplasm score by a factor, e.g., of <1, e.g., from 0 to 0.3, optionally 0.1. For example, if not found in the cytoplasm the unpenalized essentialness score is 0 and if found in the cytoplasm, the unpenalized essentialness score is 1; with the optional penalty score (e.g., of X) applied, the cytoplasm score would be 0 if not found in the cytoplasm (0*X) or X if found in the cytoplasm (1*X); with X=0.1, for example, the cytoplasm score would be 0 or 0.1.

In some cases, whether or not a protein is known to be found within the cytoplasm is reported in the literature, e.g., The UniProt Consortium, “UniProt: the Universal Protein Knowledgebase in 2023,” Nucleic Acids Res. 51: D523-D531 (2023) (www.uniprot.org), e.g., ftp.uniprot.org/pub/databases/uniprot/current_release/knowledgebase/taxonomic_divisions/uniprot_sprot_human.xml.gz.

### Ligandability Score

In some cases, calculating a ligandability score (i.e., between 0 and 1) is carried out as described above.

### Interaction Partners

In some cases, the methods described herein comprise methods for identifying protein(s) and/or protein domain(s) predicted to interact with an E3 ligase substrate receptor protein surface or neosurface, optionally an E3 ligase substrate receptor protein listed in Table 1.

In some cases identifying protein(s) and/or protein domain(s) predicted to interact with an E3 ligase substrate receptor protein surface or neosurface comprises: a) providing data comprising structural features, optionally molecular surface features of a plurality of surface patches, each of which represents a region on a respective protein molecular surface; and, based on the structural features, identifying a first set of protein(s) comprising surfaces similar to the surface or neosurface of a known or suspected E3 ligase substrate receptor protein, using a machine learning algorithm trained on structural features of interacting pairs of protein surface patches; b) identifying a second set of protein(s) known or suspected to interact with the first set of protein(s), and optionally identifying a set of protein domain(s) representing the second set of protein(s); d) optionally filtering the second set of protein(s) or the set of protein domain(s); and e) determining that one or more of second set of protein(s), the filtered second set of protein(s), the set of protein domain(s), or the set of filtered protein domain(s) is predicted to interact with the known or suspected E3 ligase substrate receptor protein surface or neosurface.

In some cases, identifying a first set of protein(s) comprises: generating embeddings for each of the surface patches by processing the data comprising the set of surface features using an embedding neural network to generate an embedding of the surface patches in an embedding space; and determining, for the surface patch, a surface/neosurface similarity score; and based on the surface/neosurface score, including or excluding the corresponding protein from the first set. In some cases, the similarity score is calculated as described in PCT/US2022/050242 or PCT/US2022/050275, which are hereby incorporated by reference in their entirety, which are attached as Appendix A and B, respectively.

In some cases, identifying a first set of protein(s) comprises identifying a set of proteins having or predicted to have a degron of the E3 ligase substrate receptor protein, (e.g., an E3 ligase substrate receptor protein predicted to be programmable as described herein), e.g., as described in PCT/US2022/050242 or PCT/US2022/050275, which are hereby incorporated by reference in their entirety, which are attached as Appendix A and B, respectively.

In some cases, including or excluding is based on a rank and/or threshold score.

In some cases, identifying a second set of protein(s) comprises searching a database (e.g., PDB or the like).

### Testing

In some cases, the methods described herein comprise testing or having tested protein(s), e.g., predicted programmable E3 ligase substrate receptor proteins and/or their corresponding known or predicted neosubstrates, in an E3 ligase substrate detection assay. In some cases, the assay is carried out in the absence of a binding modulator of the E3 ligase. In some cases, the assay is carried out in the presence of a binding modulator of the E3 ligase.

E3 ligase substrate detection assays are described, for example, in Liu et al., “Assays and Technologies for Developing Proteolysis Targeting Chimera Degraders,” Future Medicinal Chemistry 12 (12): 1155-79 (2020).

E3 ligase substrate detection assays include, for example, binding/ternary binding affinities and ternary complex formation assays used to profile, for example, ternary complex formation, population, stability, binding affinities, cooperative or kinetics such as fluorescence polarization (FP) assay, an amplified luminescent proximity homogenous assay (ALPHA), time-resolved fluorescence energy transfer assay (TR-FRET), isothermal titration calorimetry (ITC), surface plasma resonance (SPR), bio-layer interferometry (BLI), nano-bioluminescence resonance energy transfer (nano-BRET), size exclusive chromatography (SEC), crystallography, co-immunoprecipitation (Co-IP), mass spectrometry (MS), and protein-fragment complementation (e.g., NanoBiT®). See, e.g., Liu et al., 2020.

E3 ligase substrate detection assays include, for example, protein ubiquitination assays. See, e.g., Liu et al., 2020.

E3 ligase substrate detection assays include, for example, target degradation assays such as immunoassays, reporter assays, mass spectrometry (MS), protein degradation-based phenotypic screening such as amplified luminescent proximity homogenous assay (ALPHA), bio-layer interferometry (BLI), cellular thermal shift assay (CETSA), co-immunoprecipitation (Co-IP), cryogenic electron microscopy (Cryo-EM), differential scanning fluorimetry (DSF), fluorescence polarization (FP), isothermal titration calorimetry (ITC), microscale thermophoresis (MST), NanoLuc binary technology (Nano-BiT), nano-bioluminescence resonance energy transfer (BRET), surface plasma resonance (SPR), time-resolved fluorescence energy transfer (TR-FRET), tandem ubiquitin-binding entities-amplified luminescent proximity homogenous and enzyme-linked immunosorbent assay (TUBE-ALPHALISA), and tandem ubiquitin-binding entities-dissociation-enhanced lanthanide fluorescent immunoassay (TUBE-DELFIA). See, e.g., Liu et al., 2020.

In some cases, the E3 ligase substrate detection assay is a proximity assay. In some cases, the E3 ligase substrate detection assay is a binding assay. In some cases, the E3 ligase substrate detection assay is a degradation assay.

In some cases, the proximity assay is a homogeneous time resolved fluorescence (HTRF) assay. In some cases, the proximity assay is a quantitative proteomics assay. In some cases, the proximity assay is a biotinylation assay, e.g., a promiscuous biotinylation assay.

In some cases, the degradation assay is a High efficiency Binary Technology (HiBiT) assay.

In some cases, the degradation assay is a quantitative proteomics assay.

In some cases, the E3 ligase substrate detection assay is a yeast-2-hybrid system. See, e.g., Kohalmi et al., “Identification and Characterization of Protein Interactions Using the Yeast-2-Hybrid System,” In: Gelvin S. B., Schilperoort R. A. (eds) Plant Molecular Biology Manual. Springer, Dordrecht (1998). In some cases, the E3 ligase substrate detection assay is a yeast-3-hybrid system. See, e.g., Glass et al., “The Yeast Three-Hybrid System for Protein Interactions,” Methods Mol. Biol 1794:195-205 (2018).

In some cases, the E3 ligase substrate detection assay is a genomic construct based method, e.g., as described in Sievers et al., “Defining the Human C2H2 Zinc Finger Degrome Targeted by Thalidomide Analogs through CRBN,” Science 362(6414):eaat0572 (2018).

In some cases, the E3 ligase substrate detection assay is an indirect screen, e.g., to detect changes in gene and/or protein expression.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for calculating a programmability score for a known or suspected E3 ligase substrate receptor protein, the method comprising:
a) providing data comprising a first set of structural features, optionally molecular surface features representing a putative interaction region on a protein known or suspected to be an E3 ligase substrate receptor protein, optionally a protein listed in Table 1; and, based on the first set of structural features, calculating a PPI propensity score for the protein, optionally using a machine learning algorithm trained on structural features of known or predicted protein-protein interaction surfaces;
b) providing data comprising a second set of structural features, optionally molecular surface features representing a putative pocket region on the protein, and, based on the second set of structural features, calculating a ligandability score for the protein, optionally using a machine learning algorithm trained on structural features features of known or predicted high affinity small molecule pockets;
c) based on the PPI propensity score and the ligandability score, calculating a programmability score for the known or suspected E3 ligase substrate receptor protein;
d) optionally identifying the presence or absence of a cysteine residue within the putative pocket region; and
e) optionally, classifying the E3 ligase substrate receptor protein as programmable or not based on the programmability score and optionally the presence or absence of a cysteine residue within the putative pocket region.

2. The method of claim 1, wherein calculating a PPI propensity score comprises:
generating an embedding for the putative interaction region by processing the data comprising the first set of structural features using an embedding neural network to generate an embedding of the putative interaction region in an embedding space; and
determining, for the putative interaction region, the likelihood of being involved in a protein-protein interaction.

3. The method of claim 1 or claim 2, wherein calculating a ligandability score comprises:
generating an embedding for the putative pocket region by processing the data comprising the first set of structural features using an embedding neural network to generate an embedding of the putative pocket region in an embedding space; and
determining, for the putative pocket region, the likelihood of being a ligand binding pocket.

4. The method of any one of claims 1-3, wherein classifying comprises comparing the programmability score to a threshold and, if a) the threshold is satisfied and b) optionally if a cysteine is present, classifying the E3 ligase substrate receptor protein as programmable; else, classifying the E3 ligase substrate receptor protein as not programmable.

5. The method of claim 4, wherein the threshold is a programmability score for a known E3 ligase substrate receptor protein, optionally CRBN.

6. A method for calculating a desirability score for a known or suspected E3 ligase substrate receptor protein, the method comprising:
a) providing data comprising a first set of structural features, optionally molecular surface features representing a putative interaction region on a protein known or suspected to be an E3 ligase substrate receptor protein, optionally a protein listed in Table 1; and, based on the first set of structural features, calculating a PPI propensity score for the protein, optionally using a machine learning algorithm trained on structural features of known or predicted protein-protein interaction surfaces;
b) providing data comprising a second set of structural features, optionally molecular surface features representing a putative pocket region on the protein, and, based on the second set of structural features, calculating a ligandability score for the protein, optionally using a machine learning algorithm trained on structural features features of known or predicted high affinity small molecule pockets;
c) calculating an activity score for the protein;
d) calculating an essentialness score for the protein;
e) calculating a cytoplasm score for the protein;
f) calculating an expression score for the protein; and
c) based on the PPI propensity score, the ligandability score, the activity score, the essentialness score, and the cytoplasm score, calculating a desirability score for the known or suspected E3 ligase substrate receptor protein;
d) optionally identifying the presence or absence of a cysteine residue within the putative pocket region; and
e) optionally, classifying the E3 ligase substrate receptor protein as desirable or not based on the desirability score and optionally the presence or absence of a cysteine residue within the putative pocket region.

7. The method of claim 6, wherein calculating a PPI propensity score comprises:
generating an embedding for the putative interaction region by processing the data comprising the first set of structural features using an embedding neural network to generate an embedding of the putative interaction region in an embedding space; and
determining, for the putative interaction region, the likelihood of being involved in a protein-protein interaction.

8. The method of claim 6 or claim 7, wherein calculating a ligandability score comprises:
generating an embedding for the putative pocket region by processing the data comprising the first set of structural features using an embedding neural network to generate an embedding of the putative pocket region in an embedding space; and
determining, for the putative pocket region, the likelihood of being a ligand binding pocket.

9. The method of any one of claims 6-8, wherein calculating an activity score for the protein comprises assigning a numeric value based on the count of known interactions.

10. The method of claim 9, wherein the numeric value assigned is 0.0 if the count is 0, 0.1 if the count is >0 and <=10, and 0.2 if the count is >10.

11. The method of any one of claims 6-10, wherein calculating an essentialness score for the protein comprises classifying the protein as essential or not, assigning a value of 0 if essential and 1 if not essential, and optionally applying a penalty.

12. The method of claim 11, comprising applying a penalty by multiplying by a factor of from 0.0 to 0.3, optionally 0.1.

13. The method of any one of claims 6-12, wherein calculating a cytoplasm score for the protein comprises classifying the protein as known to be present in the cytoplasm or not, assigning a value of 0 if not known to be in the cytoplasm and 1 if known to be in the cytoplasm, and optionally applying a penalty.

14. The method of claim 13, comprising applying a penalty by multiplying by a factor of from 0.0 to 0.3, optionally 0.1.

15. The method of any one of claims 6-14, wherein calculating an expression score comprises assigning a numeric value based on the level of known expression.

16. The method of claim 15, wherein the numeric value assigned is 0.4 if the expression level is high, 0.2 if the expression level is medium, 0.1 if the expression level is low, and 0 if expression is not detected.

17. The method of claim 15 or claim 16, wherein the level of known expression is the average of expression in all tissues.

18. The method of any one of claims 6-17, wherein classifying comprises comparing the desirability score to a threshold and, if a) the threshold is satisfied and b) optionally if a cysteine is present, classifying the E3 ligase substrate receptor protein as desirable; else, classifying the E3 ligase substrate receptor protein as not desirable.

19. The method of claim 18, wherein the threshold is a programmability score for a known E3 ligase substrate receptor protein, optionally CRBN.

20. A method for identifying protein(s) and/or protein domain(s) predicted to interact with an E3 ligase substrate receptor protein surface or neosurface, optionally an E3 ligase substrate receptor protein listed in Table 1, optionally a known or suspected E3 ligase substrate receptor protein classified by the method of any one of claims 1-19, the method comprising:
a) providing data comprising structural features, optionally molecular surface features of a plurality of surface patches, each of which represents a region on a respective protein molecular surface; and, based on the structural features, identifying a first set of protein(s) comprising surfaces similar to the surface or neosurface of a known or suspected E3 ligase substrate receptor protein, using a machine learning algorithm trained on structural features of interacting pairs of protein surface patches;
b) identifying a second set of protein(s) known or suspected to interact with the first set of protein(s), and optionally identifying a set of protein domain(s) representing the second set of protein(s);
d) optionally filtering the second set of protein(s) or the set of protein domain(s); and
e) determining that one or more of second set of protein(s), the filtered second set of protein(s), the set of protein domain(s), or the set of filtered protein domain(s) is predicted to interact with the known or suspected E3 ligase substrate receptor protein surface or neosurface.

21. The method of claim 20, wherein identifying a first set of protein(s) comprises:
generating embeddings for each of the surface patches by processing the data comprising the set of surface features using an embedding neural network to generate an embedding of the surface patches in an embedding space; and
determining, for the surface patch, a surface/neosurface similarity score; and
based on the surface/neosurface score, including or excluding the corresponding protein from the first set.

22. The method of claim 21, wherein including or excluding is based on a rank and/or a threshold score.

23. The method of any one of claims 20-22, wherein identifying a second set of protein(s) comprises searching a database.

24. The method of any one of the preceding claims, wherein the molecular surface features comprise geometric and/or chemical features.

25. The method of claim 10, wherein the geometric features are selected from the group consisting of shape index, distance-dependent curvature, geodesic polar coordinates, radial (angular) coordinates, and combinations thereof.

26. The method of claim 10, wherein the chemical features are selected from the group consisting of hydropathy index, continuum electrostatics, location of free electrons, location of free proton donors, and combinations thereof.

27. The method of any one of the preceding claims, wherein the machine learning algorithm comprises a geometric deep learning model.

28. The method of claim 13, wherein the geometric deep learning model is a three-dimensional convolutional neural network.

29. The method of any one of the preceding claims, wherein the method is performed at least in part by one or more computers.

30. The method of any one of the preceding claims, further comprising testing or having tested the E3 ligase substrate receptor protein or protein(s) and/or protein domain(s) predicted to interact with an E3 ligase substrate receptor protein surface or neosurface in an E3 ligase substrate detection assay, with or without a binding modulator.

31. The method of claim 16, wherein the E3 ligase substrate detection assay is selected from the group consisting of a proximity assay, a binding assay, and a degradation assay.

32. A system comprising:
one or more computers; and
one or more storage devices communicatively coupled to the one or more computers, wherein the one or more storage devices store instructions that, when executed by the one or more computers, cause the one or more computers to perform operations comprising the method of any one of the preceding claims.

33. One or more non-transitory computer storage media storing instructions that when executed by one or more computers cause the one or more computers to perform operations comprising the method of any one of the preceding claims.
